(19) United States
(12) Patent Application Publication
(10) Pub. No.: US 2025/0147056 A1
(43) Pub. Date: May 8, 2025

(54) MODULARIZED ASSAY-SPECIFIC SOFTWARE INTERFACE

(71) Applicant: Fluxergy Inc., Irvine, CA (US)

(72) Inventors: Woojun Chang, Irvine, CA (US); Kirithika Sathyamoorthy, Los Angeles, CA (US); Yungkai Kyle Lai, Irvine, CA (US); Farzad Izadi Kharazi, Rancho Santa Margarita, CA (US)

(21) Appl. No.: 18/584,686

(22) Filed: Feb. 22, 2024

Related U.S. Application Data
(60) Provisional application No. 63/597,255, filed on Nov. 8, 2023.

(51) Int. Cl. G01N 35/00; G01N 35/10; G05B 19/042

(52) U.S. Cl. CPC G01N 35/00871; G01N 35/00613; G01N 35/00712; G01N 35/1072; G05B 19/042; G01N 2035/00326; G01N 2035/00881; G01N 2035/0091; G01N 2035/1032

(57) ABSTRACT

An assay analyzer measures data to evaluate whether a sample contains a certain material. Multiple assays may be performed in parallel, sometimes by the same analyzer with multiple assays on a single card, or by multiple analyzers run by a single computer. In a control computer each assay has an assay module instance to perform calculations upon the raw data measured by the analyzer to produce a final result. Each of these assay module instances operates independently and separately without influencing the calculations of the other assay module instances.

### CROSS-REFERENCE TO RELATED APPLICATION

This application is a non-provisional application of, and claims priority to, U.S. Provisional Patent Application No. 63/597,255, entitled “Modularized Assay-Specific Software Interface”, filed on Nov. 8, 2023, the entire disclosure of which is hereby incorporated by reference in its entirety.

### BACKGROUND

The present application relates to software used for analyzing data from assays to measure the presence or absence of certain materials within a subject or within an area. Example assays may be used with environmental, human, animal, biological, or other materials.

Conventional assay analysis systems utilized in a biochemical and/or biomedical environment typically perform a single assay at a time. For example, a conventional assay analysis system may receive a test card on which a sample has been provided. The conventional assay analysis system utilizes various hardware components to analyze the sample based on the type of assay being performed. Once the assay is complete, the assay analysis system may perform a similar assay or a different assay on another test card onto which another sample has been provided.

In such cases with multiple analyses being performed on the same computer or device at roughly the same time, then the software performing the analysis should not be permitted to interfere with or affect the analysis of the other assays.

Accordingly, a need arises for a modularized assay-specific software interface.

### SUMMARY

Aspects of the disclosure relate to systems and methods for determining a final result associated with each of two or more assays. The system includes an analyzer capable of analyzing a first assay and a second assay and a control computer which itself comprises a processor and memory accessible by the processor for operating a host program, a first assay module instance, and a second assay module instance. Together the analyzer, the host program and the assay module instances perform operations. The host program initiates the first assay on the analyzer and the first assay module instance on the control computer. The host program initiates the second assay on the analyzer and the second assay module instance on the control computer. The analyzer performs the first assay and the second assay. The analyzer collects a first raw data associated with the first assay and a second raw data associated with the second assay. In an example, the collection of the first raw data associated with the first assay and the second raw data associated with the second assay occurs in parallel or substantially in parallel. The host program transfers the raw data from the analyzer to the associated assay module instance (i.e. the first raw data is transferred to the first assay module instance and the second raw data is transferred to the second assay module instance). Each of the first assay module instance and the second assay module instance calculates from the raw data an intermediate result and from the intermediate result determines a final result. Each assay module instance transmits to the host program its determined final result. The host program stores both final results and displays both final results on a results screen.

The system may further comprise a scanner such as a barcode scanner or a QR code scanner or an rf ID tag scanner for automatic inclusion of additional information related to the assay. The additional information may comprise a test start configuration, a test result configuration, and a hardware instrumentation configuration. Example assays comprise at least one of a polymerase chain reaction (PCR) test, lateral flow assay (LFA), an antibody test, a colorimetric test, a turbidity test, a viscosity test, a cytometry test, or a chemical test.

### BRIEF DESCRIPTION OF THE DRAWINGS

So that the manner in which the above recited features of the present application can be understood in detail, a more particular description of the application, briefly summarized above, may be had by reference to examples, some of which are illustrated in the appended drawings. It is to be noted, however, that the appended drawings illustrate only typical examples of this application and the application may admit to other equally effective examples.

FIG. 1 illustrates a computing device for interacting with an analyzer.

FIG. 2 illustrates the computing device interacting with an analyzer.

FIG. 3 illustrates two types of test cards: a single test card and a multi-test card.

FIG. 4 illustrates independent assay module instances.

FIG. 5 illustrates an assay analyzer with multiple test cards with multiple test modalities interacting with multiple assay module instances.

FIG. 6 illustrates the computing device interacting with a multi-test card in an analyzer with a single assay module instance.

FIG. 7 illustrates the computing device interacting with a multi-test card in an analyzer with two assay module instances.

FIG. 8 illustrates the flow of information amongst the analyzer, the computing device, and the software components.

FIG. 9 illustrates an alternate method of operating multiple tests on one test card using multiple assay module instances.

FIG. 10 illustrates the computing device interacting with two test cards each with multiple assays.

Other features will be apparent from the Detailed Description that follows.

### DETAILED DESCRIPTION

In the following detailed description of the preferred examples, reference is made to the accompanying drawings, which form a part hereof, and within which are shown by way of illustration specific examples by which aspects of the application may be practiced. It is to be understood that other examples may be utilized and structural changes may be made without departing from the scope of the application. Electrical, mechanical, logical, and structural changes may be made to the examples without departing from the spirit and scope of the present teachings. The following detailed description is therefore not to be taken in a limiting sense, and the scope of the present disclosure is defined by the appended claims and their equivalents.

As used herein, “in parallel” means that a time period in which one resource item is in use overlaps entirely with a time period in which the other resource item is in use. Additionally, when two of the two or more resource items are used “substantially in parallel”, this means that a time period in which one resource item is in use overlaps, at least partially, with a time period in which the other resource item is in use. When two of the two or more resource items are used “in series”, a time period in which one resource item is in use arises prior to or subsequent to a time period in which the other resource item is in use.

It is sometimes advantageous to perform multiple assays or the same assay multiple times simultaneously. In such occurrences multiple test cards may be used in multiple analyzers, all of which may be controlled by a single computer. In another example the test card may be appropriately designed for more than a single assay. In cases in which different assays are performed on the same card usually a single computer controls the processes. In order to assure the reproducibility of the tests, it is imperative that the software performing the analysis on each of the different assays not interfere with any of the other analysis being performed.

For example, conventional in vitro diagnostics (IVD) test platforms typically only perform one test modality. (In this contact, modality means a type of test. For example, a test platform may perform both a PCR test and an LFA. Each of these assays is considered a modality or operation.) Some diagnostics test platforms can perform a small number of different tests, but not at the same time. Other diagnostics test platforms can perform only a limited number of different tests at one time. Overall, testing capabilities of conventional diagnostic test platforms are limited by the time and space requirements for multiple diagnostic tests. This often results in limited diagnostic information and negative patient outcomes.

In a clinical setting, some test results from conventional platforms require a subsequent confirmation or reflex test before a diagnosis or clinical outcome is decided. These confirmation or reflex tests can be of a different modality than the initial test, for instance a PCR test after an LFA test, or a second PCR after a first PCR to make false positives or false negatives less probable. In other situations, a combination of tests is required to determine a diagnosis, a clinical outcome, or a conclusive result. These scenarios require managing multiple conventional POC platforms or testing services from centralized laboratories. As a result, total cost of diagnosis and time to result is greater than that attributed by an individual, conventional POC test.

The present disclosure relates to assays or tests with one or more modalities. In a single mode operation an assay card is inserted into an apparatus which performs an analysis. A computer program, when executed by a computing device, performs one or more operations. These operations may include, but are not limited to, opening valves, admitting reagents, heating a sample, cooling a sample, illuminating a sample, detecting illumination from a sample, and additional operations, to run the assay on a sample in the assay card. If the analyzing apparatus is capable of performing multiple assays, for instance a PCR assay and a cytometry assay, then both of these modes/modalities would be run on the analyzer apparatus, potentially at the same time. In order for each test to run without interference from the other test and, after the test itself has been locked down (not to be changed), for instance, after having been approved by regulators, then future instances of that test must be made immutable to preserve the reproducibility of the assay. In order to facilitate such non-interference with other tests running on the same apparatus at the same time, the software which interacts with the apparatus must also be isolated from other instances of the software running other tests.

In an example, a user interface (or host program) is created which allows a user to enter relevant information for all tests being run on an apparatus, for instance by a multi-modal assay card. Since two modes may be run, each mode of operation must be approved first by regulators. If the first modality is approved first, and the second modality is approved later after changes to the computer code have been enacted, then each mode must have its own instance of the computer program to run each of the tests independently on the immutable version of the computer program. Locking down, fixing, or making the computer code immutable ensures that the test produces reliable results for all subjects whose samples are being analyzed. Confirming that the computer code for a particular test is not altered nor the results changed requires that each of the testing modalities has its own instance of computer code running and that each of the instances cannot in any way interfere with the any other instance if computer code running a different test at the same time.

In an example, a sandbox environment may be used to isolate an instance of a program to, for instance, debug the program or to identify and debug malware. In the case of an assay apparatus with more than one mode of operation, it makes more sense to have a single user interface for the user and then to spin up or run individual computer program for each of the assay/testing modes being employed. Each of the individual instances being executed must be so designed as to be unable to access any of the memory accessible by the other instances. A separate program, the user interface, is given permission to read these memory locations.

In current solutions, most human intended in vitro diagnostic (IVD) instruments modularize their software such that releasing an additional assay on the platform does not require a patch of the underlying firmware of the instrument. This makes sense, as firmware patches can carry a risk to the patient, as well as high regulatory threshold. However, there are tradeoffs for this design.

The algorithm associated with the assay remains immutable. This limits or prevents optimization of algorithm performance on a per-assay basis. For example, if one assay's biological characteristics produce a signal differing from a previously released assay on the same instrument (or the original assay released with the instrument).

Analyzing data from a different diagnostic modality is not possible. Because different analyte interrogation methods produce fundamentally different signal types, an algorithm from one modality (e.g., PCR—amplification curve analysis), cannot analyze the results from another (e.g., image cytometry—cell counting).

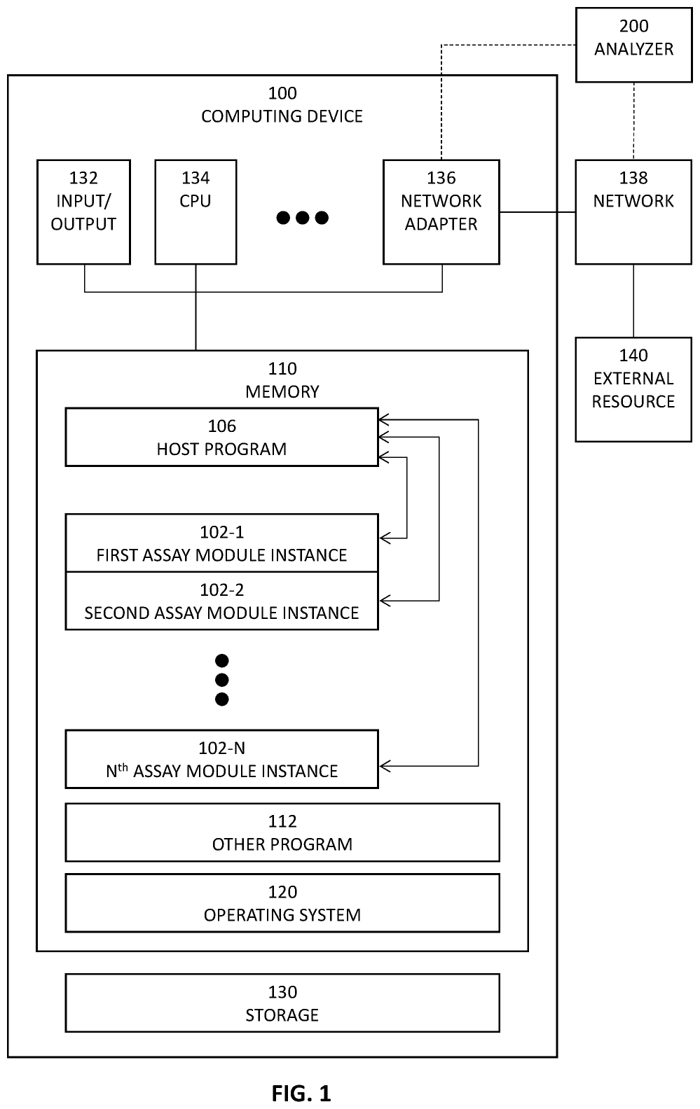

FIG. 1 illustrates a computing device 100 for use with an analyzer 200, for example an assay test platform. The computing device 100 comprises an input/output element 132, for example a keyboard, a microphone, a mouse, a display, a speaker, a scanner, a QR code scanner or the like, a central processing unit (CPU) 134, a network adapter 136, memory 110, and storage 130. In an example, a CPU 134 includes multiple processors. In an example storage 130 may comprise a hard disk drive, optical disks, a flash drive, and the like. The network adapter 136 enables communication between the computing device 100 and a network 138 or between the computing device 100 and the analyzer 200. The computing device 100 may connect to the analyzer 200 through a dedicated connection to the network adapter 136 or through the network adapter 136 to the general network 138. In another example, the analyzer 200 is a stand alone system and/or is operable separate from the computing device. However, the analyzer 200 may still be communicatively coupled to the computing device 100.

The network 138 may include an external resource 140, such as a database of recipes for an analyzer apparatus 200 or remote storage of data such as configuration files, recipe files, and the like. Within the computer memory 110 are stored programs. The programs may include an operating system 120 as well as a host program (e.g., a user interface) 106 and instances of an assay module 102-1, 102-2, . . . 102-N. In some examples, for example when the analyzer 200 is configured to perform more than one assay, then there may be multiple instances of an assay module instance 102-1, 102-2, . . . 102-N being executed concurrently by the processor 134, as detailed elsewhere in this disclosure. In some examples, a single instance of the assay module may interact with an analyzer performing multiple assays, as detailed elsewhere in this disclosure. FIG. 1 depicts three such assay module instances: a first assay module instance 102-1, a second assay module instance 102-1, and an N^{th} assay module instance 102-N. Other programs 112 may also be stored in memory for execution by the processor 134.

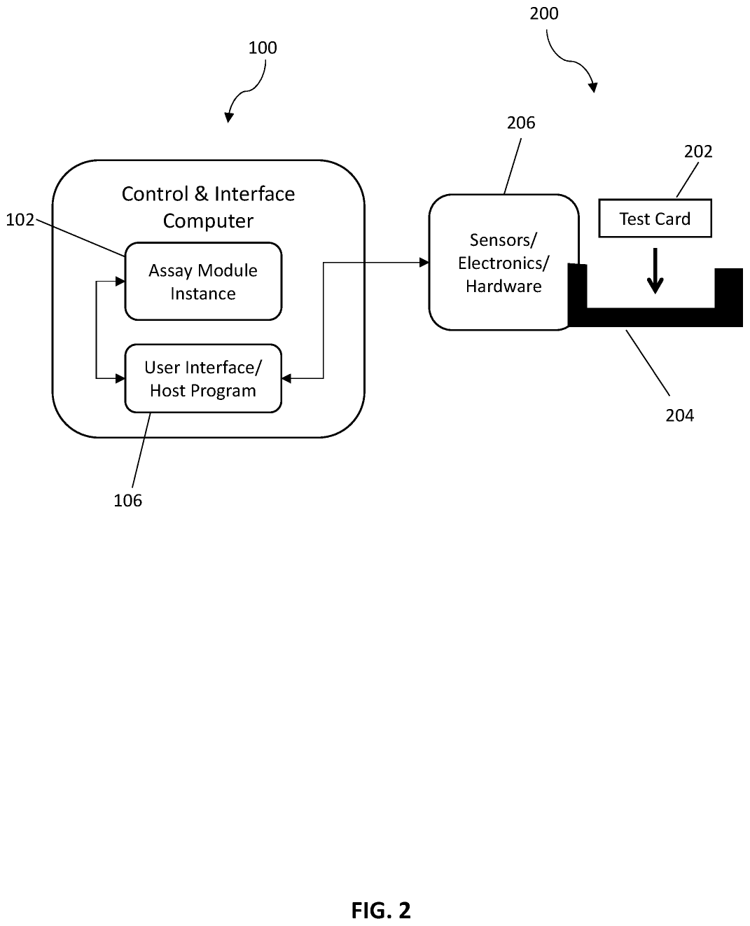

FIG. 2 illustrates an analyzer 200 along with the control computer 100. The control computer 100 has running in it, amongst other elements, an assay module instance 102 and a host program or user interface 106. The host program 106 communicates with the analyzer 200 while the assay module instance 102 communicates to and from the host program 106. The assay module instance 102 is thus isolated from directly interacting with the analyzer 200 in this example. The analyzer 200 comprises electronics and hardware including sensors 206, a test card receptacle or port 204 into which a test card 202 is inserted. The test card may be a single test card 202 comprising a single assay or a multi-test card 302 comprising two or more assays. In FIG. 2 a single test card 202 is shown.

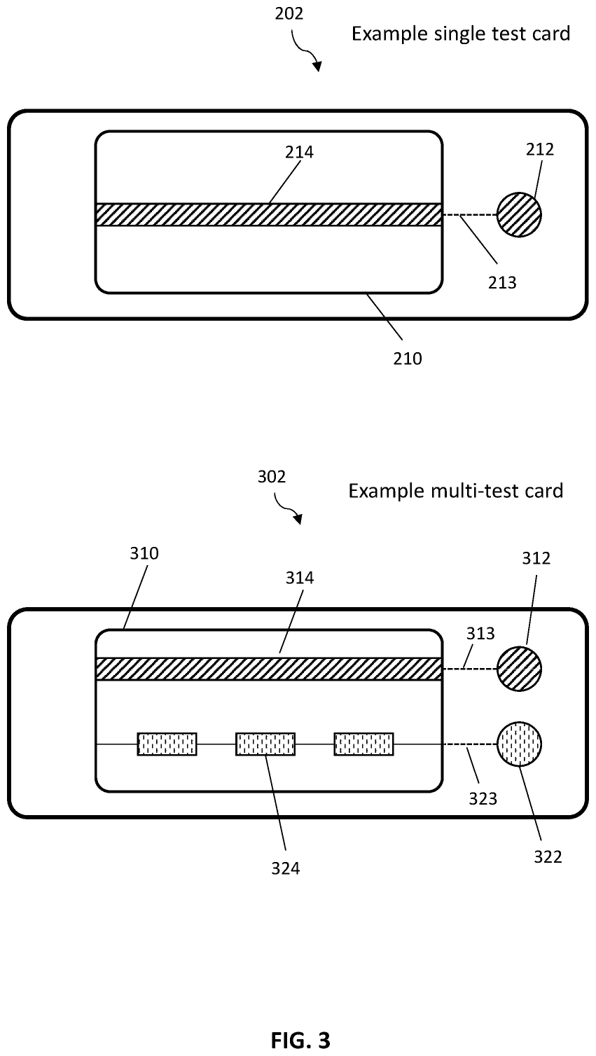

FIG. 3 schematically illustrates two types of test cards. The upper portion of the figure illustrates a single test card 202 and the lower portion of the figure illustrates a multi-test card 302. The single test card 202 comprises a test bed 210, a sample port 212, a channel 213, and a test strip 214. The channel 213 connects the sample in the sample port 212 with the test strip 214. Other portions of the single test card 202 such as heaters, sensors, valves, other channels, etc. are not shown for simplicity. The example multi-test card 302 also has a testing area 310 but comprises more than one sample port. In the example multi-test card 302 shown, there is a first sample port 312 and a second sample port 322. The test bed 310 of the multi-test card 302 may comprise a first test strip 314 for testing a sample from the first sample port 312 and a second test strip 324 for testing a sample from the second sample port 322. In an example, the first test strip 314 may be a cytometry test and the second test strip 324 may be a PCR test. The hardware for running such tests is incorporated in the test card 202, 302 itself or in the hardware 206 for the analyzer 200. In an example, the first test strip 314 may test for a first modality (e.g., LFA) on the first sample and the second test strip 324 may test for a second modality (e.g., PCR) on the second sample. While the examples depicted in FIG. 2 show a multi-test card 302 with two tests, a multi-test card 302 may have any number of tests.

The user interface for starting a test and the user interface for reporting results for different modalities are not compatible. The information required for the initiation of a PCR test is not identical to that required for the initiation of a cytometry test. Likewise, the reporting of results between the two aforementioned modalities are fundamentally different (a Ct value or viral load in the former, a count of the number and types of cells in the latter). This holds true for any two or more given modalities.

Along with the reasons noted above, combining modalities in a commercially released human-intended IVD instrument after-the-fact in current solutions is not possible. The configuration of tests the device can operate is unchangeable after regulatory approval.

A system may have several main components: the assay module package, a communication interface, a set of static configurations, and a virtual environment.

In an example, the assay module package is a software artifact which encapsulates the next three components. It can be of any format, such as zip, tar, etc.

A communication interface communicates into and out of the assay module instance from the host program or between the host program and the analyzer. By means of this communication interface, data is sent from the analyzer to the host program and from the host program into the assay module for analysis. Also by means of the communication interface, the results of analyses are transmitted out of the assay module to the host program, internal process statuses are reported, and configuration information regarding the assay being run is sent to the analyzer.

The set of configurations defines the following: User interface (UI) elements and interface logic, such as valid and invalid field values, for initiating the specific assay being run. The UI elements and interface logic such as printing options and display configurations, for reporting the results of the specific assay being run. The configuration also includes a recipe which defines the set of hardware control and measurement steps to run a specific assay.

The virtual environment may comprise two sub-programs: an algorithm and a set of business logic. The algorithm and the business logic may be written in a variety of programming languages or packages such as Python or Matlab. The algorithm may be packaged in a manner to eliminate any external dependencies. The algorithm carries out the analysis of raw data, and outputs extracted signal information or an intermediate result. The business logic may analyze the signal information or intermediate result and may supply the final result (e.g., cell count, positive/negative, etc.).

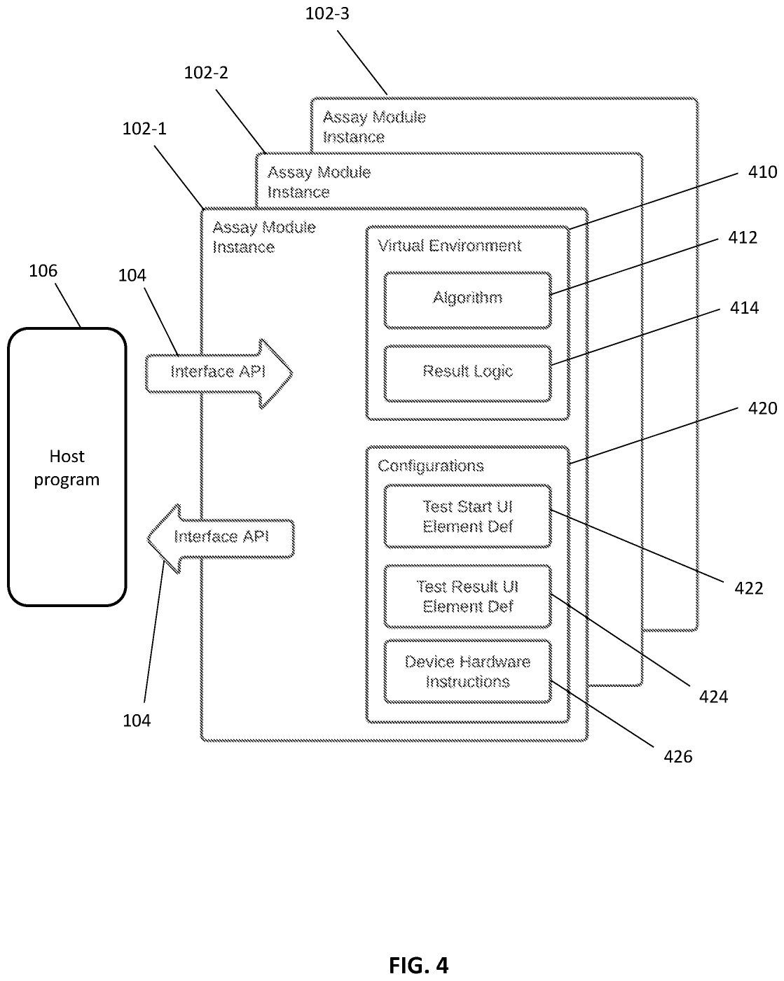

FIG. 4 illustrates three assay module instances running concurrently. The figure illustrates a first instance 102-1, a second instance 102-2, and a third instance 102-3 of an assay module being executed on a single analyzer 200 using a single computing device 100 at the same time. The assay analyzer 200 itself is not shown in this figure. In an example, the instances 102-1, 102-2, and 102-3 were started separately (e.g., in sequence) but are running concurrently (or in parallel) on the same processor, but with different memory allocations. In another example, such as for example, in a multiprocessor system, each sequence may be started at the same time and run concurrently or in parallel. An API 104 communicates between each of the instances 102 and the main user interface or host program 106. For example, the host program 106 may receive raw data from the analyzer 200. Then the API 104 may transfer the raw data from the host program 106 to the assay module instance 102. Once the assay module instance 102 has performed calculations on the raw data to produce a final result, the API 104 may transfer the final result to the host program 106. The virtual environment 410 within the assay module instance 102 may comprise an algorithm sub-module 412 for receiving raw data and processing the raw data and result logic 414 for determining a final result. The result logic 414 may determine a final result based on the processed data (and possibly also including other information). The assay module instance 102 may comprise a configurations sub-module 420 for various configurations of the analyzer and the test. For example, a configurations sub-module 420 may comprise test start user interface (UI) configuration information 422, test result UI configuration information 424, and device hardware instructions 426. Examples of some configuration files are provided in the Appendix at the end of this disclosure.

### Single Test Card, Single Analyzer, Single Assay Module Instance

In an example, as illustrated in FIG. 2, a single test card 202 may be inserted into a port 204 of the analyzer 200. For this exemplary single test card 202 only a single instance 102 of an assay module software is needed to run on the associated computer 100. In an example, the test card 202 may have a single mode of operation (e.g., a single PCR test).

### Single Test Cards, Multiple Analyzers, Multiple Assay Module Instances

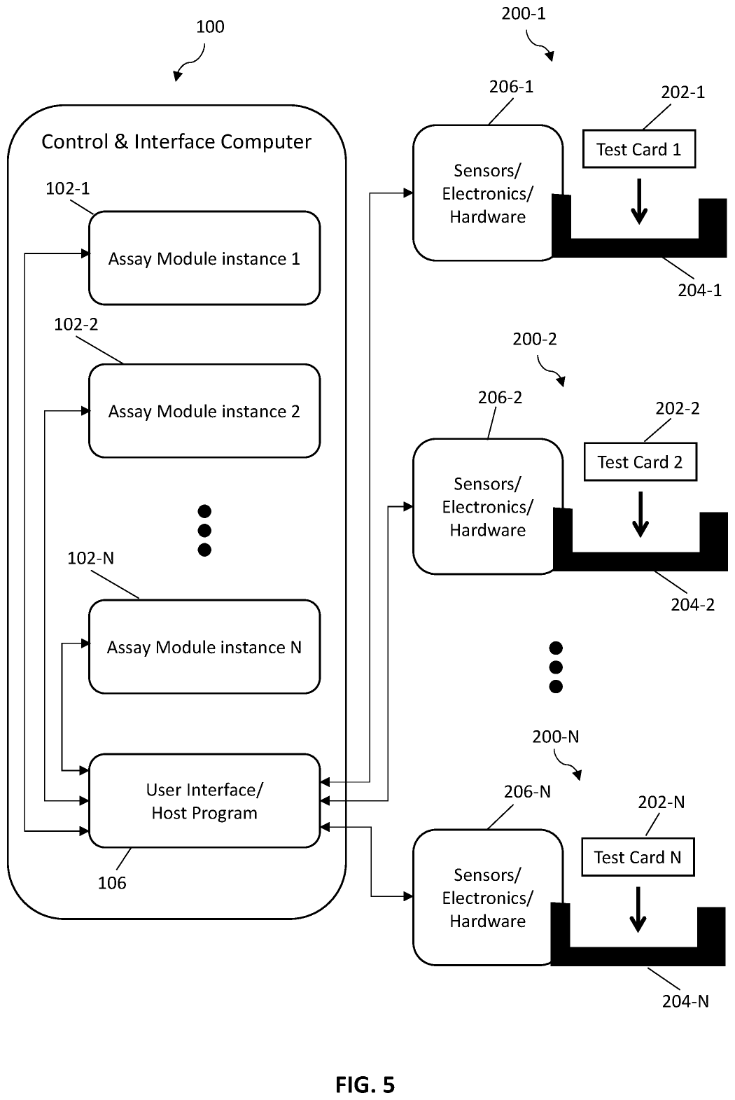

In an example, illustrated in FIG. 5, two or more analyzers 200-1, 200-2, . . . 200-N are controlled by a single computer 100. Each analyzer receives a single test card 202. For instance, the first analyzer 200-1 receives a first test card 202-1. The second analyzer 202-2 receives a second test card 202-2. The N^{th} analyzer 200-N receives the N^{th} test card 202-N. The analyzers 200-1, 200-2, . . . 200-N each have their associated hardware 206-1, 206-2 . . . 206-N and test card ports 204-1, 204-2, . . . 204-N. For each test card 204, one instance of the assay module software 102 operates on the computer. Note that the host program 106 interacts directly with each of the analyzers 200-1, 200-2, . . . 200-N to receive raw data and transfer the raw data to the associated assay module instance 102-1, 102-2, . . . 102-N. The assay module instances 102 do not interact or communicate directly with the analyzer 200. In this example, the first assay module instance 102-1 receives the raw data associated with the first analyzer 200-1 and provides a final result associated with the first analyzer 200-1 to the host program 106. In this example, the second assay module instance 102-2 receives the raw data associated with the second analyzer 200-2 and provides a final result associated with the first analyzer 200-2 to the host program 106. The host program 106 acts as an interface between the analyzers 200 and the assay module instances 102. Only a single instance of the host program 106 (e.g. Fluxergy Works) may be required in this example. The host program 106 interacts with each of the assay module instances 102 to provide each assay module instance 102 with the raw data and to receive the final results and display the final results to a user.

When the first analyzer 200-1 finishes the test(s) and has the raw data ready, the host program 106 transfers the raw data to the first assay module instance 102-1. The first assay module instance 102-1 then processes the data, determines a final result and sends the final result to the host program 106 for display to a user. In an example, the first assay module instance 102-1 may store the final result in, for instance, a specific memory address or may write the final result to a specific location on a storage medium 130 (e.g., on a hard drive). The first assay module instance 102-1 may also write the raw data, the processed data, or the final result to a storage medium 130.

In the example of having multiple analyzers a user may insert each test card 202 into each test card receptacle 204 of each analyzer 200 and initiate an assay module instance 102 to receive the data, process the data, and provide a final result. In some examples, the assay module instance 102 may be initiated first and then the test card 202 may be inserted into the receptacle 204. In other examples, the test card 202 may be inserted into the receptacle 204 first and the assay module instance 102 started second. In an example, the host program 106 may be started first and information entered (e.g., patient name, number, test name, date, location, etc.) prior to starting an instance of the assay module 102. In an example, the test card 202 may comprise a bar code, a QR code, or other code, which may be scanned automatically so that the user interface 106 recognizes the test or tests associated with the scanned test card 202 and automatically starts the assay module instance 102 (or instances) with the proper information required to run the test (e.g., recipe information for the analyzer 200 to perform a PCR assay on the sample in the scanned and inserted test card 202). In an example, once the test or tests finish, the host program 106 may pull the raw data from the analyzer 200 and send the raw data to the associated assay module instance 102. The assay module instances 102 may calculate an intermediate result by processing the data and then may return a final result to the host program 106. The host program 106 may store the result and the raw data in a storage medium 130 and may display the results to a user. Since there are multiple tests being performed, the tests may be performed in parallel, partially in parallel, partially in series, or fully in series, as specified by a user or by the test recipes themselves. The assay module instances 102-1, 102-2, . . . 102-N may also operate in parallel, partially in parallel, partially in series, or fully in series, as desired by the user or by the host program. If the control computer 100 has multiple processors, the individual assay module instances 102 may operate fully in parallel. If the control computer 100 has a single processor 134 (or fewer processors than assay module instances), then two or more assay module instances 102-1, 102-2, etc. may operate concurrently on the same processor 134.

### Multi-Test Card, Single Analyzer, Single Assay Module Instance

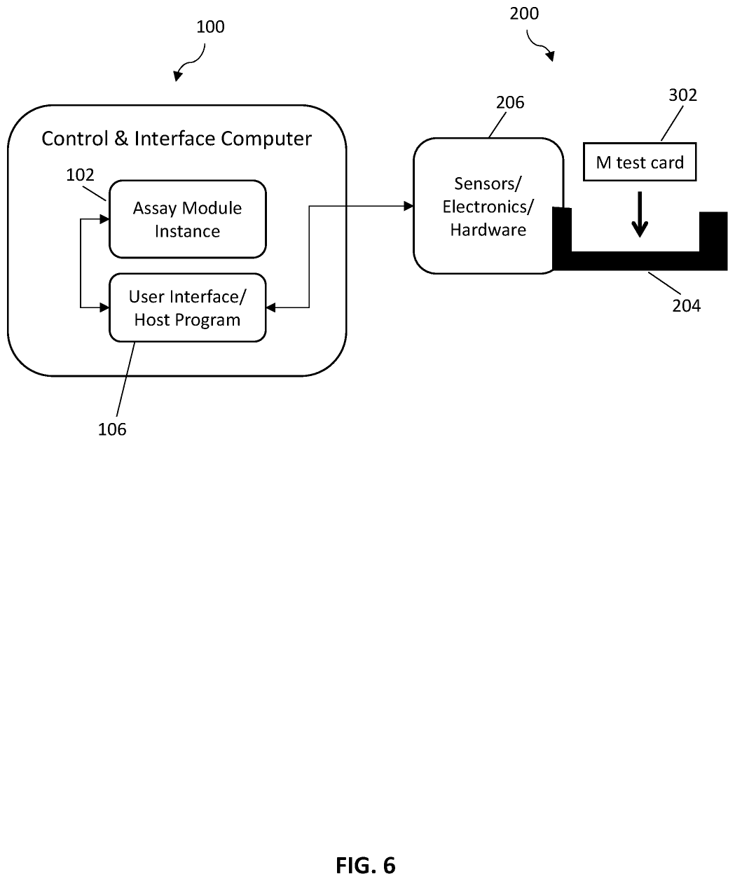

In another example, depicted in FIG. 6, a multi-test card 302 may have two or more modalities of operation (e.g., one PCR test and one cytometry test, or two PCR tests and one LFA test, etc.) In the case of a test card 302 having two or more modalities, a single assay module instance 102 may be all that is required to interact with the host program 106 which is interacting with the analyzer 200.

FIG. 6 illustrates an example of a single analyzer 200 and a single assay module instance 102 for a multi-test card 302 with two or more tests/assays/modalities. A test card may, of course, contain more than two tests, but the generality of including additional tests is unchanged from 2 to many tests. In this example, a multi-test card 302 has two tests: a first test and a second test, as illustrated in FIG. 3. The first test may comprise a first sample port 312, a first channel 313, and a first test strip 314. The second test may comprise a second sample port 322, a second channel 323, and a second test strip 324.

In the example shown in FIG. 6, the computer 100 may be running the host program 106 and the host program 106 may have already initiated the assay module instance 102. In this example, there is a single analyzer 200, with a single receptacle 204 and one multi-test card 302 which has two or more assays on it. In this example, the host program 106 may initiate any one of the tests on the multi-test card 302 or the host program 106 may initiate one or more tests on the multi-test card 302. These multiple tests may be performed in parallel, in series, or partially in parallel and partially in series depending on the precise recipe used to execute the assay. In an example, a single assay module instance 102 is all that is required to receive the raw data from each of the two tests from the host program, process the two sets of raw data, and produce at least two final results.

### Multi-Test Card, Single Analyzer, Multiple Assay Module Instances

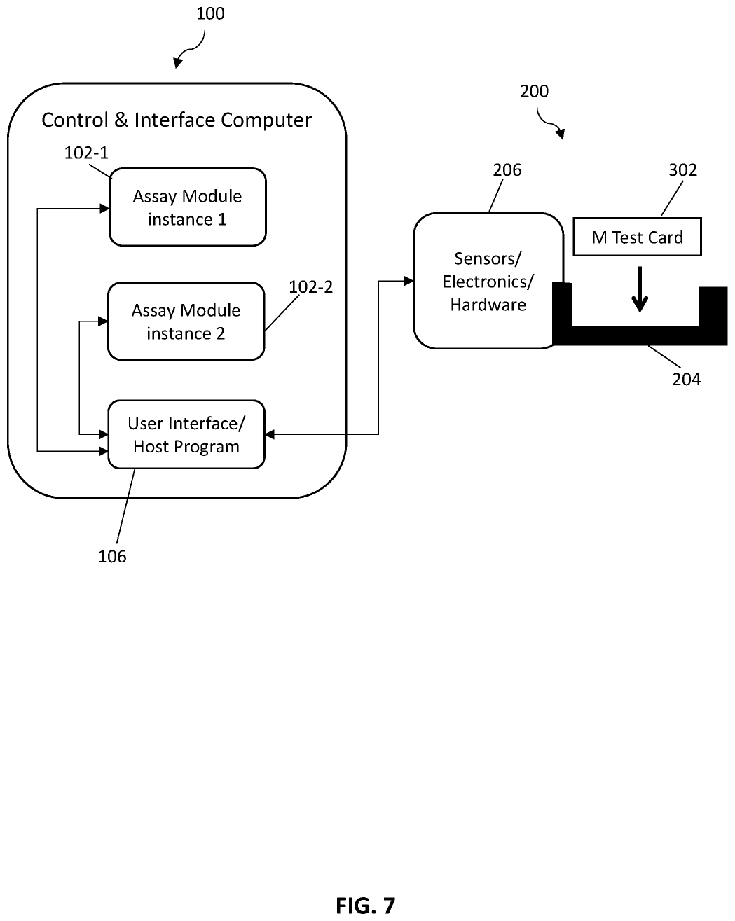

FIG. 7 illustrates another example in which one multi-test card 302 is inserted into the receptacle 204 of a single analyzer 200. The host program 106 interacts with the analyzer 200 to control the two (or more) tests and to receive the raw data that the tests produce. In this example, the first assay module instance 102-1 receives the raw data from the first test of the multi-test card 302 and the second assay module instance 102-2 receives the raw data of the second test of the multi-test card 302. Both of these assay module instances 102-1, 102-2 may process the raw data to produce a first final result and a second final result which may be reported to the user interface/host program 106. Although two assay module instances are mentioned, additional module assays may be used.

### Advantages of Isolation

There is a significant benefit to having an assay module instance 102 operate independently of the user interface/host program 106 and also independently of other assay module instances. In order to be sure that no individual test interferes with any other test (or, indeed, that no other program on the computer 100, including malware, viruses, and the like interferes with operation of the assay or interferes with analysis of the data), each assay module instance 102 can be made immutable in the same way that the analyzer 200 has hardware 206 which cannot be modified without negatively impacting the reliability of the test results. Isolating the software which analyzes the raw data allows a user to be confident of the reliability of the tests. In some circumstances, it may not be possible to modify an analyzer, or its software, for instance, after it has been approved by a regulatory body.

In this manner, therefore, the assay module instances 102-1, 102-2, . . . 102-N may each be qualified at different times and each one is “locked down” or made immutable once it has been approved by regulators. If a new test is added to a card, and the new test achieves regulatory approval with, say, an updated version of software (e.g., a newly trained machine learning model using data collected after approval of the first test, or a new random number generator is used from an update in the operating system), then the old analysis may still operate since the old analysis may operate independently of the new analysis or the new test. As noted elsewhere in this disclosure the raw data is collected only at the analyzer, and the analysis, data processing, and production of the final results only occurs in a specific assay module instance.

In the example shown in FIG. 5, multiple analyzers 200-1, 200-2, etc. can be running different assays, each of which achieved regulatory approval at a different time. The final results of the various tests will not interfere with each other because each one is analyzed by a different assay module instance 102-1, 102-2, etc. which has not been changed since regulatory approval.

Achieving isolation of the assay module instances may require multiple steps during software development. External dependencies of each of the assay module instances must be eliminated. For example, an assay module instance may call upon an external code library with advanced processing options as part of the data analysis. Since such code libraries may be updated as newer or more efficient methods become available, an external library may change over time which may, in turn, alter the results. In an example, the specific sections of code in a library accessed by an assay module instance when it passed regulatory compliance, may be written to a specific location in the storage device which is not accessible to other programs so that the libraries accessible by the assay module instance are also made immutable. In other circumstances, when calling a library, the program may access an external server which has its libraries regularly updated or changed. If the second assay module instance requires a library which has been updated after the library required by the first assay module instance, then both of these libraries could be stored on the control computer storage, but each assay module instance would only be permitted to directly access the appropriate code library for itself and not to access any other such code libraries. In an example, two assay module instances may access the same general library for some components of the analysis. If both require the same components, then they could both access the same library or they could access different librarys. For example, two or more libraries are kept separate in order to decrease a risk of unintended effects (e.g., interdependencies between assay modules). However, in an example, if one assay module instance requires some specific components from a library, then that assay module instance may access these specific components independently of any other assay module instances.

In an example, it is possible to include all the code library components (or a subset of library components), in each assay module instance as it is instantiated. In another example, a set of core library components is included in, and accessible by, each assay module instance. If each assay module instance needs to use some of the core components of the library, then the computer may store one set of such core components in a library accessible by each assay module instance. In an alternative example, core components of a library used by more than one assay module instance may be accessed by each assay module instance as needed, but specific components needed by a single assay module instance may be accessible by that specific assay module instance only.

In an example, of accessing a function or component from a code library, if the code is accessed, then the second assay module instance which uses the component must be so designed so that if the first assay module instance uses the code snippet, then the second assay module instance's intermediate results are not affected. In an example, if a first assay module instance calls for a random number from, for example, a pseudo-random number generator from the operating system 120 then subsequent outputs of the pseudo-random number generator may be influenced by the first call. In this example, if any additional random numbers are called for by a second assay module instance, then they would be influenced by the first call for a random number by the first assay module instance. In such an example, each assay module instance must be so designed that they rely not on any single output of a pseudo-random number generator, but on an ensemble of many such outputs. For example, some machine learning methods start with a random assignment of certain inputs and then iterate until the outputs do not change to within a threshold amount. If an assay module instance uses such a method, then it must call the method many times to be reasonably certain that any single random assignment does not distort the output. Thus if a second assay module instance also uses a set of random assignments, the second assay module instance must also call upon a large enough set of such assignments that the output is reasonably unlikely to be distorted by a particular set of initial conditions. In some computers, the random number generator is a hardware random number generator, for which an additional software instance would not necessarily affect the output of the random number generator, but the same logic applies to the design of the different assay module instances that enough random assignments would be required to avoid any distortion of results based on a single random number or single random assignment.

Another factor in isolating different instances of the assay module is insuring that the memory allocated to each instance is not accessible by the other instances and, indeed, not accessible by any other program 112 running on the computer, with the exception, under certain circumstances, of the host program 106 or of the operating system 120. Since the same methods are used to prevent malware or malicious users from corrupting computer functions generally, analogous techniques and methods may be employed here. For example, general computer memory allocated to each instance may be assigned randomly in blocks to prevent other programs from finding the proper memory addresses to overwrite with alternative instructions. In another example, when a program accesses a memory address to which it does not have permission, an alarm may notify the user that there has been some contamination of the process or that another program attempted to read or write to a memory address allocated to the assay module instance.

### Details of One Example

In an example, the overall flow may follow the steps from Table 1.

TABLE 1
Exemplary steps for test execution

| Step | Description |
|---|---|
| 1 | User selects test(s) (e.g., scans test barcode, types in test code(s)) |
| 2 | The host program (e.g., Fluxergy Works) loads a unique assay module instance into |
| | memory for each test selected |
| 3 | Host program pulls configuration files from storage for selected test(s) and transfers them |
| | to the analyzer |
| 4 | User provides out all additional information associated with selected test(s) |
| 5 | User inserts consumable (e.g., test card) to start test |
| 6 | Host program pulls the test recipe(s) from storage and transmits the recipe(s) to the |
| | instrument by, for example, an API. |
| 7 | Analyzer/Instrument runs test(s) and collects raw data for each selected test |
| 8 | Raw data is transmitted back to the host program (e.g., by an API) |
| 9 | Host program passes raw data to appropriate assay module instance algorithm submodule |
| 10 | Algorithm submodule processes data and sends processed data (intermediate results) to the |
| | result logic submodule |
| 11 | Result logic submodule evaluates intermediate results and produces a final result |
| 12 | Host program pulls final result from the assay module instance |
| 13 | Host program stores final result and produces a results screen for the end user |
| 14 | Assay module instance is shut down |

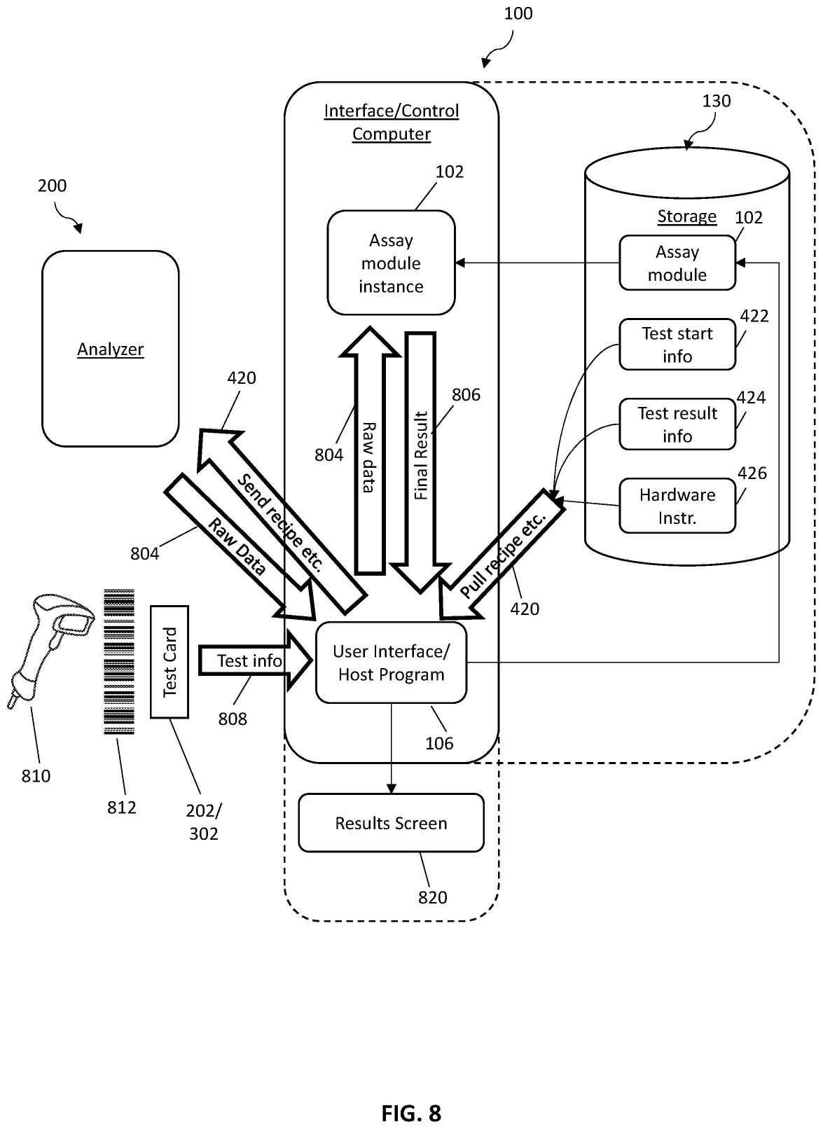

FIG. 8 illustrates some of the steps and interactions between components. The components may be separate or may be combined and accomplish the same or similar results. The components within the large, bold arrows are those data whose transfer may be facilitated by a communication interface. In an example, a single test card 202 or multi-test card 302 has a barcode 812, which is scanned by a scanner 810. The test information 808 from the card is sent to the host program/user interface 106 by an API. The host program 106 loads an assay module instance 102 from storage 130 into the computer memory 110 and starts the instance executing on the central processing unit 134. The host program 106 pulls from storage 130 the necessary configuration files 420 to perform the test. The configuration files 420 comprise the test start information 422, the test result information 424, and a hardware instrument configuration 426, such as a test recipe. The configuration files 420 are then forwarded to the analyzer 200 by the host program 106. In an example, the analyzer 200 conducts the test (or tests) and sends the raw data 804 to the host program 106 once each test is completed. In another example, the host program 106 may query the analyzer 200 for the raw data 804 or a status check after a threshold amount of time has elapsed. In another example, the analyzer 200 sends raw data 804 or a status update to the host program 106 after a threshold amount of time has elapsed. Although time is specifically mentioned, a query or a status check may be performed based, at least in part, on an event, a milestone, or a received input. For example, a query or a status check may be performed when a particular module is accessed or reached. In another example, a query or a status check may be performed when a particular amount of data and/or results are received. In yet another example, a query or status check may be performed when an error condition (e.g., a failed test) is detected.

The host program 106 sends the raw data 804 to the assay module instance 102 by an API. The assay module instance 102 performs the algorithm 412 on the raw data 804 to produce processed data or an intermediate result. The result logic 414 is applied to the processed data to produce a final result 806. The final result 806 is sent to or shared with the host program 106. The host program 106 then displays the final result 806 to a user on, for example, a results screen 820. In another example, the final result 806 may be transmitted to a user's personal electronic device rather than displayed on a results screen 820 on the interface computer 100. In another example, the host program 106 may write the final result 806 to storage 130. The storage 130 may be nearby or even inside the control computer 100 or the storage 130 may be remote from the control computer 100. For this reason, both the results screen 820 and the storage 130 are shown in a dotted line as being part of the control computer 100 since these items may be physically separated or remote from the control computer 100 and the analyzer 200.

Note that multiple assay module instances can be running at the same time, independent of each other (except for hardware resources such as RAM or CPU utilization). The host program 106 (e.g., Fluxergy Works) becomes a harness which spools up and shuts down assay module instances 102. The host program's 106 only functionality being that which reads data from an assay module, coordinates assay modules, and allows users to interact with test results. In an example, the assay module instance 102 may write the final result 806 to a hard drive or other storage medium 130 and then share with the host program 106 the location of the stored final result 806. In another example, the assay module instance 102 may store the final result 806 in memory 130 (e.g. RAM) and may send the memory address directly to the host program 106. In this example, the host program 106 may write the final result 806 to storage 130.

In this disclosure, each new assay and/or modality can be loaded, modified, and unloaded independently of any other. There are no dependences between assay modules of differing assays. Additionally, assay modules for a given assay do not share a runtime environment when operating. In this way, the instrument 200 and UI 106 do not require patches when a new assay is produced, even if the assay is of a different modality. This method of isolating each instance of an assay module 102 has regulatory benefits in that no patch is needed on the firmware or UI to analyze the raw data 804 and produce a final result 806. All regulatory risk can be isolated to the assay module 102, which is validated alongside the assay undergoing regulatory approval.

Additionally, the instrument can be guaranteed to operate in the same manner regardless of assay or modality, reducing patient risk when introducing new products to market.

Other benefits include evaluating the algorithm with in-silico analysis which matches the final product more closely, rather than a ported version of the algorithm contained on the instrument (which may be in a different programming language more suited for instrument control).

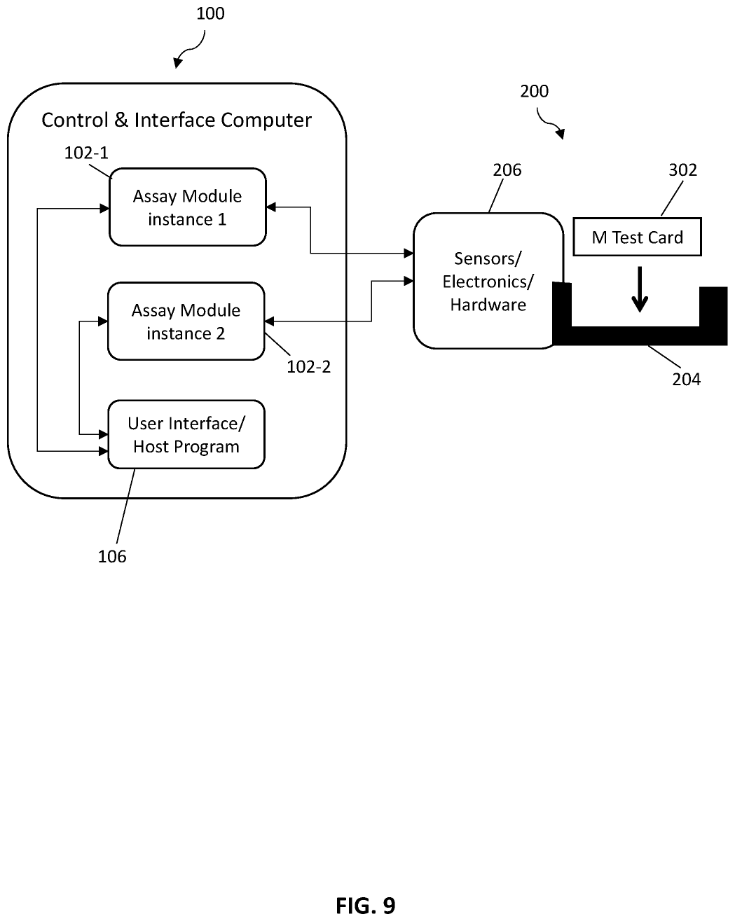

FIG. 9 illustrates and alternative example of the system. The difference between FIG. 7 and FIG. 9 is that in FIG. 9 the assay module instances 102-1 and 102-2 each interact with the analyzer 200 rather than the host program 106 interacting with the analyzer. In this alternative example, a first assay module instance 102-1 receives from the analyzer 200 a first set of raw data (not shown), calculates a first final result (not shown), and shares the first final result with the host program 106. The second assay module instance 102-2 receives a second set of raw data from a second test on the multi-test card 302. The second assay module instance 102-2 processes the second set of raw data and produces a second final result which it shares with the host program 106. In alternative example of FIG. 9, the assay module instances 102 are not isolated from the analyzer 200 itself as in the cases described elsewhere in this disclosure. While the alternative example will work, the disadvantage to permitting interactions between the assay module instance 102 and the analyzer itself are that modifications or updates to any part of the analyzer 200 or the analyzer hardware 206 may lead to changes in the final result 806 of the test or tests. In the case when the assay module instance 102 is not isolated from the analyzer apparatus 200 changes or alterations in the sensors, electronics or hardware 206 of the analyzer 200 or even in firmware updates of the hardware 206 might influence the fidelity of the final results 806 reported to the user.

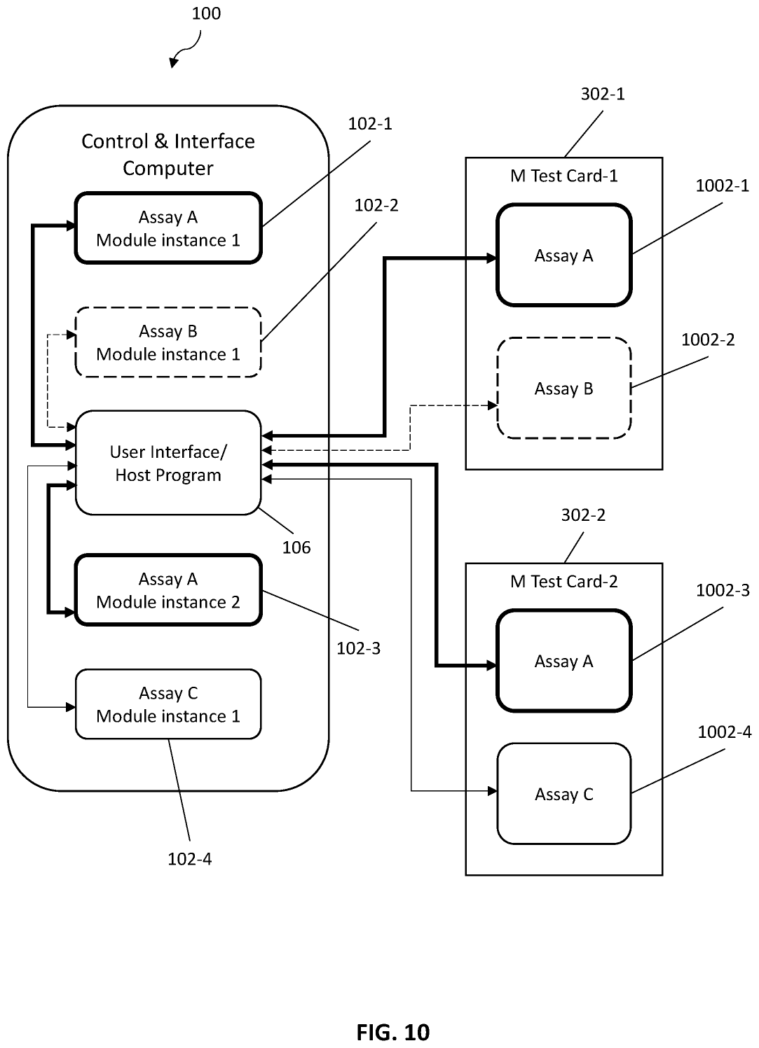

FIG. 10 illustrates and example example of the system. FIG. 10 has omitted the analyzer hardware and the test slot to better illustrate how the system and method may function. In this example, the analyzer has multiple cards and therefore multiple sets of hard, much as illustrated in FIG. 5; however, in this example, the control computer 100 interacts with two analyzers, each of which can perform multiple tests. These tests reside on a first multiple test card 302-1 and a second multiple test card 302-2. The first multiple test card 302-1 has two tests: Assay A 1002-1 and Assay B 1002-2. The second multiple test card 302-2 also has two tests: Assay A 1002-3 and Assay C 1002-4. Thus in this example Assay A is being performed twice, once on each test card. To emphasize the distinctness of the interactions, both the first Assay A 1002-1 and the second Assay A 1002-3 are shown in bold outline. Assay B 1002-2 has a dashed line for its communication and Assay C 1002-4 has a regular thickness line. In this example, the user interface 106 interacts with the two multiple test cards 1002-1, 1002-2 via the analyzer (not shown) to start four assay module instances: Assay A Module Instance 1 102-1, Assay B Module Instance 1 102-2, Assay A Module Instance 2 102-3, and Assay C Module Instance 1 102-4. Thus, even though the Assay is being performed on both the first multiple test card 302-1 and the second multiple test card 302-2, different instances are called, one for each assay or test on a test card. In this example, Assay A Module Instance 1 102-1 receives raw data only from Assay A 1002-1 on multiple test card 1 302-1. Analogously, Assay A Module Instance 2 102-2 receives raw data only from Assay A 1002-3 on multiple test card 2 302-2. Thus, even though the same analysis may occur in each of the instances of Assay A 102-1 and 102-3, they will not be in communication with each other and the analysis thus occurs completely independently.

While the methods and systems described in this disclosure are not limited by the types of assays being performed by the analyzer, some example assays are shown in Table 2 below.

TABLE 2
Example Assay Types

Example Assays
Nucleic Acid Amplification
Cytometry
Clinical Chemistry
Immunochemistry

The flowchart and block diagrams in the Figures illustrate the architecture, functionality, and operation of possible implementations of systems, methods, and computer program products according to various examples. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or that carry out combinations of special purpose hardware and computer instructions.

Although specific examples have been described, it will be understood by those of skill in the art that there are other examples that are equivalent to the described examples. Accordingly, it is to be understood that the examples described are not to be limited by the specific illustrated examples, but only by the scope of the appended claims.

### APPENDIX

### Exempl Start Test Screen Schema

```
{
  “type”: “object”,
  “properties”: {
    “assay_id”: {
      “type”: “integer”
    },
    “assay_name”: {
      “type”: “string”
    },
    “input_fields”: {
      “type”: “object”,
      “properties”: {
        “test_name”: {
          “type”: “object”,
          “properties”: {
            “location”: {
              “type”: “array”,
              “items”: [
                  {
                    “type”: “integer”
                  },
                  {
                    “type”: “integer”
                  }
                ]
              },
              “color”: {
                “type”: “string”
            },
            “size”: {
              “type”: “integer”
            }
          },
          “required”: [
            “location”,
            “color”,
            “size”
          ]
        },
        “sample_id”: {
          “type”: “object”,
          “properties”: {
            “location”: {
              “type”: “array”,
              “items”: [
              {
                “type”: “integer”
                },
                {
                  “type”: “integer”
                }
              ]
            },
            “color”: {
              “type”: “string”
            },
            “size”: {
              “type”: “integer”
            }
          },
          “required”: [
            “location”,
            “color”,
            “size”
          ]
        }
      },
      “required”: [
        “test_name”,
        “sample_id”
      ]
    },
    “display_test_template”: {
      “type”: “boolean”
    },
    “notes_field”: {
      “type”: “object”,
      “properties”: {
        “location”: {
          “type”: “array”,
          “items”: [
            {
              “type”: “integer”
            },
            {
              “type”: “integer”
            }
          ]
        },
        “color”: {
          “type”: “string”
        },
        “size”: {
          “type”: “integer”
        }
      },
      “required”: [
        “location”,
        “color”,
        “size”
      ]
    },
    “background_color”: {
      “type”: “string”
    },
    “logo_file_location”: {
      “type”: “string”
    }
  },
  “required”: [
    “assay_id”,
    “assay_name”,
    “input_fields”,
    “display_test_template”,
    “notes_field”,
    “background_color”,
    “logo_file_location”
  ]
}
```

### Start Test Screen Example

```
{
  “assay_id”: 12339348,
  “assay_name”: “CoVID-19 Singleplex”,
  “input_fields”: {
    “test_name”: {
      “location”: [
        24,
        40
      ],
      “color”: “#800080”,
      “size”: 64
    },
    “sample_id”: {
      “location”: [
        60,
        40
      ],
      “color”: “#800080”,
      “size”: 128
    }
  },
  “display_test_template”: false,
  “notes_field”: {
    “location”: [
      24,
      80
    ],
    “color”: “#FFFFFF”,
    “size”: 256
  },
  “background_color”: “#E3DBF6 ”,
  “logo_file_location”: “../imgs/Fluxergy_logo.png”
}
```

### Exempl Results Test Screen Schema

```
{
  “type”: “object”,
  “properties”: {
    “assay_id”: {
      “type”: “integer”
    },
    “assay_name”: {
      “type”: “string”
    },
    “time_params”: {
      “type”: “object”,
      “properties”: {
        “test_start”: {
          “type”: “integer”
        },
        “test_end”: {
          “type”: “integer”
        },
        “timezone”: {
          “type”: “string”
        }
      },
      “required”: [
        “test_start”,
        “test_end”,
        “timezone”
      ]
    },
    “platform_params”: {
      “type”: “object”,
      “properties”: {
        “firmware_ver”: {
          “type”: “string”
        },
        “fluxergyworks_ver”: {
          “type”: “string”
        },
        “device_serial”: {
          “type”: “integer”
        },
        “fluxergyworks_serial”: {
          “type”: “integer”
        }
      },
      “required”: [
        “firmware_ver”,
        “fluxergyworks_ver”,
        “device_serial”,
        “fluxergyworks_serial”
      ]
    },
    “fluxergyworks_type”: {
      “type”: “string”
    },
    “display_test_template”: {
      “type”: “boolean”
    },
    “notes_field”: {
      “type”: “object”,
      “properties”: {
        “location”: {
          “type”: “array”,
          “items”: [
            {
              “type”: “integer”
            },
            {
              “type”: “integer”
            }
          ]
        },
        “color”: {
          “type”: “string”
        },
        “size”: {
          “type”: “integer”
        }
      },
      “required”: [
        “location”,
        “color”,
        “size”
      ]
    },
    “background_color”: {
      “type”: “string”
    },
    “logo_file_location”: {
      “type”: “string”
    }
  },
  “required”: [
    “assay_id”,
    “assay_name”,
    “time_params”,
    “platform_params”
    “fluxergyworks_type”,
    “display_test_template”,
    “notes_field”,
    “background_color”,
    “logo_file_location”
  ]
}
```

### Results Test Screen Example

```
{
  “assay_id”: 12339348,
  “assay_name”: “CoVID-19 Singleplex”,
  “time_params”: {
    “test_start”: 5679993,
    “test_end”: 5680252,
    “timezone”: “UTC+8”
  },
  “platform_params”: {
    “firmware_ver”: “1.3.5”,
    “fluxergyworks_ver”: “3.6.8”,
    “device_serial”: 12345678,
    “fluxergyworks_serial”: 5678801
  },
  “fluxergyworks_type”: “Developer”,
  “display_test_template”: false,
  “notes_field”: {
    “location”: [
      24,
      80
    ],
    “color”: “#FFFFFF”,
    “size”: 256
  },
  “background_color”: “#E3DBF6 ”,
  “logo_file_location”: “../imgs/Fluxergy_logo.png”
}
```

What is claimed is:

1. A system for receiving raw data related to two or more assays and for determining final results associated with each of the two or more assays comprising:
a first assay performed by an analyzer;
a second assay performed by the analyzer;
a control computer comprising a processor and memory accessible by the processor for operating a host program, a first assay module instance, and a second assay module instance to perform operations comprising:
initiating, by the host program, the first assay on the analyzer and the first assay module instance on the control computer;
initiating, by the host program, the second assay on the analyzer and the second assay module instance on the control computer;
performing, by the analyzer, the first assay and the second assay;
collecting, by the analyzer, a first raw data associated with the first assay and a second raw data associated with the second assay;
transferring, by the host program, the raw data from the analyzer to the associated assay module instance;
the first assay module instance and the second assay module instance, each operating independently of each other and any other assay module instances, for:
calculating from the raw data an intermediate result;
determining a final result based on the intermediate result; and
transmitting to the host program the final result;
upon receipt of each of the final results, associated with each assay module instance, the host program storing each of the final results and displaying the final result on a results screen.

2. The system of claim 1, wherein the system further comprises a scanner to scan a code with assay information associated with the first assay and with the second assay.

3. The system of claim 2, wherein the scanner comprises at least one of an optical scanner or a radio frequency scanner and the code comprises at least one of an rf ID tag, a barcode or a QR code.

4. The system of claim 2, wherein the assay information comprises a test start configuration, a test result configuration, and a hardware instrumentation configuration.

5. The system of claim 1, wherein the first assay comprises at least one of a polymerase chain reaction (PCR) test, lateral flow assay (LFA), an antibody test, a colorimetric test, a turbidity test, a viscosity test, a cytometry test, or a chemical test.

6. A system for receiving raw data related to two or more assays and determining final results associated with each of the two or more assays comprising:
a first assay performed by a first analyzer;
a second assay performed by a second analyzer;
a control computer comprising memory and a processor for operating a host program, a first assay module instance, and a second assay module instance to perform operations comprising:
initiating, by the host program, the first assay on the first analyzer and the first assay module instance on the control computer;
initiating, by the host program, the second assay on the second analyzer and the second assay module instance on the control computer;
performing, by the first analyzer, the first assay;
performing, by the second analyzer, the second assay;
collecting, by the first analyzer, a first raw data associated with the first assay;
collecting, by the second analyzers, a second raw data associated with the second assay;
transferring, by the host program, the first raw data from the first analyzer to the first assay module instance;
transferring, by the host program, the second raw data from the second analyzer to the second assay module instance;
the first assay module instance, operating independently of the second assay module instance to:
calculate from the first raw data a first intermediate result;
determine a first final result based on the first intermediate result; and
transmit to the host program the first final result;
the second assay module instance, operating independently of the first assay module instance to:
calculate from the second raw data a second intermediate result;
determine a second final result based on the second intermediate result; and
transmit to the host program the second final result;
storing, by the host program, the first final result and the second final results; and
displaying, by the host program, the first final result and the second final result on a results screen.

7. The system of claim 6, wherein the system further comprises a scanner to scan a code with assay information associated with the first assay and with the second assay.

8. The system of claim 7, wherein the scanner comprises at least one of an optical scanner or a radio frequency scanner and the code comprises at least one of an rf ID tag, a barcode or a QR code.

9. The system of claim 7, wherein the assay information comprises a test start configuration, a test result configuration, and a hardware instrumentation configuration.

10. The system of claim 6, wherein the first assay comprises at least one of a polymerase chain reaction (PCR) test, lateral flow assay (LFA), an antibody test, a colorimetric test, a turbidity test, a viscosity test, a cytometry test, or a chemical test.

11. A method performed in a control computer with a host program, a first assay module instance, and a second assay module instance for receiving raw data related to two or more assays and for determining final results associated with each of the two or more assays comprising:
initiating, by the host program, the performance of a first assay on the analyzer;
initiating, by the host program, the first assay module instance on the control computer;
initiating, by the host program, the performance of a second assay on the analyzer;
initiating, by the host program, the second assay module instance on the control computer;
performing, by the analyzer, the first assay;
performing, by the analyzer, the second assay;
collecting, by the analyzer, a first raw data associated with the first assay and a second raw data associated with the second assay;
transferring, by the host program, the first raw data from the analyzer to the first assay module instance;
transferring, by the host program, the second raw data from the analyzer to the second assay module instance;
the first assay module instance and the second assay module instance, each operating independently of each other and any other programs, adapted to:
calculate, from the first raw data or the second raw data, a first intermediate result or a second intermediate result;
determine a first final result or a second final result based on the first intermediate results or the second intermediate result; and
transmit to the host program the first final result or the second final result;
upon receipt of the first final result and receipt of the second final result, the host program storing the first final result and the second final result; and
displaying for a user the first final result and the second final result on a results screen.

12. The method of claim 11, wherein the method further comprises scanning a code with assay information associated with the first assay and with the second assay.

13. The system of claim 12, wherein the scanning comprises using at least one of an optical scanner or a radio frequency scanner and the code comprises at least one of an rf ID tag, a barcode or a QR code.

14. The system of claim 12, wherein the assay information comprises a test start configuration, a test result configuration, and a hardware instrumentation configuration.

15. The system of claim 11, wherein the first assay comprises at least one of a polymerase chain reaction (PCR) test, lateral flow assay (LFA), an antibody test, a colorimetric test, a turbidity test, a viscosity test, a cytometry test, or a chemical test.

16. A method performed in a control computer with a host program, a first assay module instance, and a second assay module instance for receiving raw data related to two or more assays and for determining final results associated with each of the two or more assays comprising:
initiating, by the host program, the first assay on a first analyzer;
initiating, by the host program, the first assay module instance on the control computer;
initiating, by the host program, the second assay on a second analyzer;
initiating, by the host program, the second assay module instance on the control computer;
performing, by the first analyzer, the first assay;
performing, by the second analyzer, the second assay;
collecting, by the first analyzer, a first raw data associated with the first assay;
collecting, by the second analyzer, a second raw data associated with the second assay;
transferring, by the host program, the first raw data from the first analyzer to the first assay module instance;
transferring, by the host program, the second raw data from the second analyzer to the second assay module instance;
the first assay module instance, operating independently of the second assay module instance to:
calculate from the first raw data a first intermediate result;
determine a first final result based on the first intermediate result; and
transmit to the host program the first final result;
the second assay module instance, operating independently of the first assay module instance to:
calculate from the second raw data a second intermediate result;
determine a second final result based on the second intermediate result; and
transmit to the host program the second final result;
storing, by the host program, the first final result and the second final result; and
displaying, by the host program, the first final result and the second final result on a results screen.

17. The method of claim 16, wherein the method further comprises a scanning a code with assay information associated with the first assay and with the second assay.

18. The method of claim 17, wherein scanning comprises using at least one of an optical scanner or a radio frequency scanner and the code comprises at least one of an rf ID tag, a barcode or a QR code.

19. The method of claim 17, wherein the assay information comprises a test start configuration, a test result configuration, and a hardware instrumentation configuration.

20. The method of claim 16, wherein the first assay comprises at least one of a polymerase chain reaction (PCR) test, lateral flow assay (LFA), an antibody test, a colorimetric test, a turbidity test, a viscosity test, a cytometry test, or a chemical test.
